(19) United States
(12) Patent Application Publication
(10) Pub. No.: US 2011/0276141 A1
(43) Pub. Date: Nov. 10, 2011

(54) INTERVERTEBRAL SPACER, SYSTEM AND METHOD TO DISTRACT ADJACENT VERTEBRAE AND INSERT A SPACER

(76) Inventor: Alexandre Caratsch, Trelex (CH)

(21) Appl. No.: 13/021,794

(22) Filed: Feb. 7, 2011

(30) Foreign Application Priority Data
May 5, 2010 (CH) 00689/10
Jan. 17, 2011 (CH) 0073/11

(51) Int. Cl. A61F 2/44

(52) U.S. Cl. 623/17.16

(57) ABSTRACT

Intervertebral spacer being a longitudinal body with a cross-section that has a long axis and a short axis, at least one component of which may be deployed following an axis that is essentially perpendicular to the longitudinal body's long axis and to the axis of the spine.

### TECHNICAL FIELD

The present invention relates to the medical field, and more particularly to a intervertebral spacer and a system for the distraction of adjacent vertebrae and the insertion of the intervertebral spacer.

### BACKGROUND OF THE INVENTION

Intervertebral spinal cages and other inserts are known to treat certain conditions of the human spine, such as degenerative disc disease. Their function is to keep adjacent vertebrae apart and stabilize the vertebral segment pending fusion of said adjacent vertebrae.

A common problem with the existing intervertebral cages and spacers is that they have a bulky width, as they need to remain laterally stable once they are implanted. However, their width is an obstacle to their insertion via a minimally invasive surgical procedure.

In U.S. Pat. No. 6,290,724, Marino describes a method for separating and stabilizing adjacent vertebrae using an insert with curved lateral cam surfaces that is pushed laterally into the intervertebral space until its final position, and thereafter rotated 90° laterally to separate adjacent vertebrae through the cam effect of its lateral surfaces, whereupon the insert anchors into the endplates of the vertebral bodies. In US 2002/0055745, McKinley describes a method to insert a bone block between adjacent vertebrae using an inserter separating adjacent vertebrae in the same way as the insert described in U.S. Pat. No. 6,290,724 does separates adjacent vertebrae. In US 2004/0088054, Berry describes a cage that has laterally expanding wings that fit in an inner chamber formed within the central body of the cage.

### SUMMARY OF THE INVENTION

The present invention relates to an intervertebral spacer, with a longitudinal body, the width of which is substantially smaller than its height so that its cross-section has an oval shape or the shape of a race-track. One or both of the flanks of the longitudinal body may be deployed laterally such that the width of the spacer is increased. The shape of the spacer allows the separation of adjacent vertebrae after the insertion of the tip of the spacer with its smaller section perpendicular to the axis of the spine followed by the lateral rotation of the spacer of an angle close to 90°. The spacer is thereafter pushed in its upright position (with its smaller cross-section parallel to the axis of the spine) to its final position in the intervertebral space, where one or more of its flanks are deployed laterally.

In one preferred embodiment of the invention, the deployment of the flank may be achieved by inflation of a balloon in the flank or by the dilatation of specific material integrated in the flank. In another embodiment, the deployment of the flank may be achieved by the lateral unfolding of one or several crutches, which may be portions of one or both flanks of the longitudinal body, which may be achieved by the rotation of such portions around the axis of one or more hinges linking that portion or those portions to the longitudinal body. In another embodiment, the lateral displacement of the portion of one flank is achieved by it being pushed forward and sliding against a curved path built in the flank.

In another preferred embodiment, the spacer may be placed in a sheath, the cross section of which is essentially oval or in the shape of a race-track. This system enables the insertion of the tip of the sheath between adjacent vertebrae with its smaller section perpendicular to the axis of the spine followed by the lateral rotation of the sheath of an angle close to 90°. The sheath is thereafter pushed in its upright position (with its smaller cross-section parallel to the axis of the spine) close to the final position considered for the spacer in the intervertebral space. The spacer is thereafter pushed through the inside of the sheath and released into the intervertebral space, where one or several of its flanks are deployed according this invention. A variation of this embodiment consists of having the spacer housed in the sheath during the insertion, rotation and push of the sheath in the intervertebral space.

Another embodiment of the invention is to cause the lateral deployment of the longitudinal body's flank or flanks by narrowing the space between the distal and proximal ends of the longitudinal body. This may be achieved with a hinge placed two portions of one flank, and other hinges connecting each respective portion of that flank to the tip and posterior portions of the longitudinal body, respectively. When the tip and posterior portions of the longitudinal body are brought closer, for instance through the turning of a conveying screw, or the tensioning of a cable, connecting both said portions, the flank with the hinge deploys laterally and an angle is created around the hinge. In yet another embodiment of the invention, this deployment may also be achieved via a flank made of flexible material, so that the narrowing of the space between the distal and proximal portions of the longitudinal body causes that flank in flexible material to bulge outwardly.

The deployment of the flanks under any of the embodiments increases the perimeter of the contact surfaces with the two respective endplates which stabilizes the spacer in its lateral axis. The various embodiments of the invention may be applied to spacers for all sorts of surgical approaches: postero-lateral, transforaminal, lateral, antero-lateral and anterior.

The hinges described in the embodiments may also operate as articulations in more than one dimension, allowing the longitudinal body and its deployable flanks to maintain motion between the adjacent vertebrae.

The characteristics of the invention will appear more clearly in the descriptions of the preferred embodiments of the invention which will be made by way of example and shall not be limitative of the scope of the invention.

### BRIEF DESCRIPTION OF THE DRAWINGS

FIG. 1a is a perspective view of the spacer, with non-deployed flanks

FIG. 1b represents the same view of the spacer as in FIG. 1b, but with deployed flanks through inflation or swelling

FIG. 1c represents the same view as in FIG. 1b, but with deployed flanks through inflation or swelling for a spacer for transforaminal or lateral surgical approaches

FIG. 2a represents a cross-section of a vertebral segment with the tip of the spacer inserted with the longer axis of its cross-section parallel to the endplates of the vertebrae

FIG. 2b represents the same view as in FIG. 2a, but after rotation of 90° of the spacer

FIG. 2c represents the same view as in FIG. 2b, but after deployment of the flanks of the spacer

FIG. 2d represents the same view as in FIG. 2c, but with deployed flanks of a spacer for transforaminal or lateral surgical approaches

FIG. 3a represents a spacer with a folded lateral crutch

FIG. 3b represents the same view as in FIG. 3a, but with deployed lateral crutch

FIG. 4 represents a perspective view of a spacer housed in a sheath

FIG. 5a represents a cross-section of a vertebral segment with the tip of the sheath inserted with the longer axis of its cross-section parallel to the endplates of the vertebrae

FIG. 5b represents a view in axial plane of a vertebra with the tip of the sheath inserted with its wider dimension parallel to the endplate

FIG. 5c represents the same view as in FIG. 5a after a 90° rotation of the sheath.

FIG. 5d represents the same view as in FIG. 5b after rotation of the sheath

FIG. 5e represents a view in axial plane of a vertebra with the sheath progressing in its upright position along a straight trajectory

FIG. 5f represents a view in axial plane of a vertebra after partial removal of the sheath and partial delivery of the spacer with one crutch deploying

FIG. 5g represents the same view as in FIGS. 5a and 5c with the spacer with a laterally deployed crutch

FIG. 5h represents a view in axial plane of a vertebra with two spacers with laterally deployed crutches

FIG. 5i represents a view in axial plane of a vertebra with one spacer for unilateral approach having two laterally deployed crutches

FIG. 6a represents a view in axial plane of a vertebra with a sheath in an upright position delivering one spacer in memory shape alloy along a <<T>> trajectory

FIG. 6b represents the same view as in FIG. 6a but with one laterally deployed crutch

FIG. 7a represents a perspective view of one spacer with one flank whose anterior and posterior portions may be contracted thus deploying laterally one flank connected with hinges

FIG. 7b represents a view from top of the spacer in FIG. 7a during lateral deployment of one articulated flank

FIG. 7c represents the same view as in FIG. 7b, after deployment of the articulated flank.

FIG. 7d represents a view from top of a spacer with deployed articulated half-flanks

FIG. 7e represents a view from top of a spacer with symmetrically deployed flanks

FIG. 8 represents a view from top of a spacer with one flank of flexible material bulging laterally

FIG. 9a represents a spacer with a lateral crutch that may deploy by sliding against one flank

FIG. 9b represents the same view as in FIG. 9c with the deployed sliding crutch

### DESCRIPTION OF THE EMBODIMENTS OF THE INVENTION

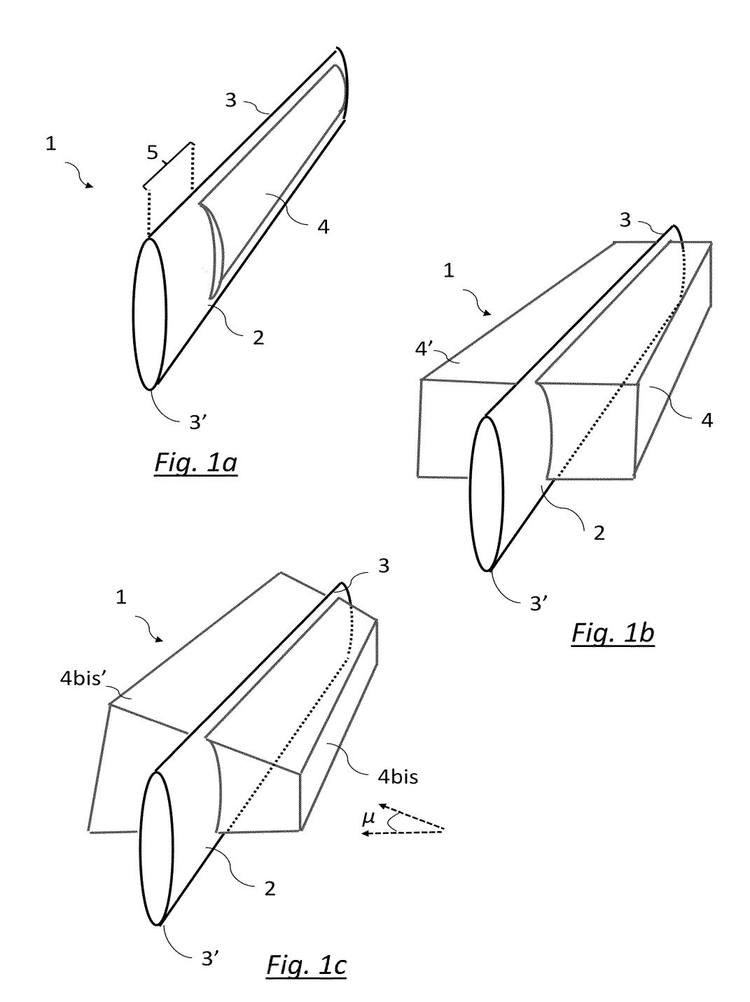

According to a first embodiment of the invention, described in FIGS. 1a, 1b and 1c, spacer 1 comprises of a longitudinal body 2 which has an oval or ellipsoidal cross section or a cross-section in the shape of a race-track, with two edges 3, 3′, and two flanks 4, 4′ which are deployable by inflation. Longitudinal body 2 has a tip 5. Flanks 4, 4′ may be made in extensible material, such as of polymers, or of a synthetic or organic or semi-synthetic material. They may also be balloons that are inflatable by air, liquids or bone cements. FIG. 1b represents a spacer 1 with deployed flanks configured for posterior or postero-lateral surgical approaches. This spacer 1 may also have a cross-section with a receding height (not represented in a figure) so as to give a lordotic angle to the longitudinal body 2. FIG. 1c represents a spacer 1 with flanks 4bis, 4bis′ deployed is such different heights as to offer a lateral lordosis described as <<μ>>: this type of spacers may thus be appropriate for transforaminal, lateral and antero-lateral approaches. Spacer 1 is attached in its posterior part to a longitudinal inserter (not represented) by any possible technical means, which inserter allows to laterally rotate and push spacer 1.

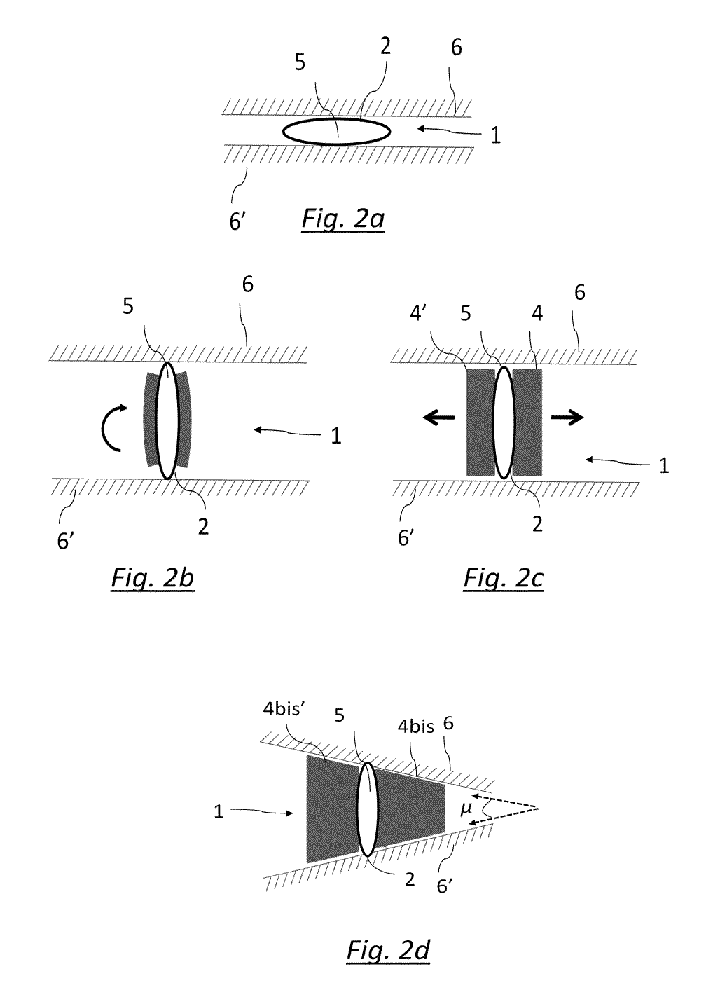

The insertion method is described in FIGS. 2a to 2d, which represents a section view of a segment of two adjacent vertebrae 6, 6′ and the tip of spacer 5. FIG. 2a shows the vertebral segment 6, 6′ with the tip 5 of the longitudinal body 2 inserted with its larger cross-section in a parallel plane with the slightly distracted endplates of vertebrae 6, 6′. FIG. 2b represents the same vertebral segment after a 90° lateral rotation of the longitudinal body 2. The intervertebral space has been distracted by the tip 5 through the lever effect of the rotation force applied to the longitudinal body 2 via the inserter. Longitudinal body 2 is now positioned with its larger cross-section perpendicular to the endplates 6, 6′. Longitudinal body 2 is pushed forward in a straight trajectory. FIG. 1c describes longitudinal body 2 at its final location, and the deployment of its flanks 4, 4′ to constitute a deployed spacer 1. If the flanks are balloons, they are inflated and/or filled with air, liquid, viscous material or bone cement at this stage. The flanks may also be made of another material such as dehydrated and compacted foam, which may swell and become rigid through contact with adjoining human tissue. They may also be made of a soft matrix that becomes more rigid with the adding of a catalytic substance. Spacer 1 represented in FIG. 2c may be used for posterior and postero-lateral surgical approaches. FIG. 2d represents a spacer 1 for unilateral approaches, such as transforaminal or lateral, and which corresponds to the spacer represented in FIG. 1c after insertion between two adjacent vertebrae 6, 6′.

According to another aspect of the invention, the flanks 4, 4′, 4bis, 4bis′ may have a height exceeding the height of longitudinal body 2 and serve as buffer and may avoid or mitigate the contact between edges 3, 3′ of the longitudinal body and endplates 6, 6′.

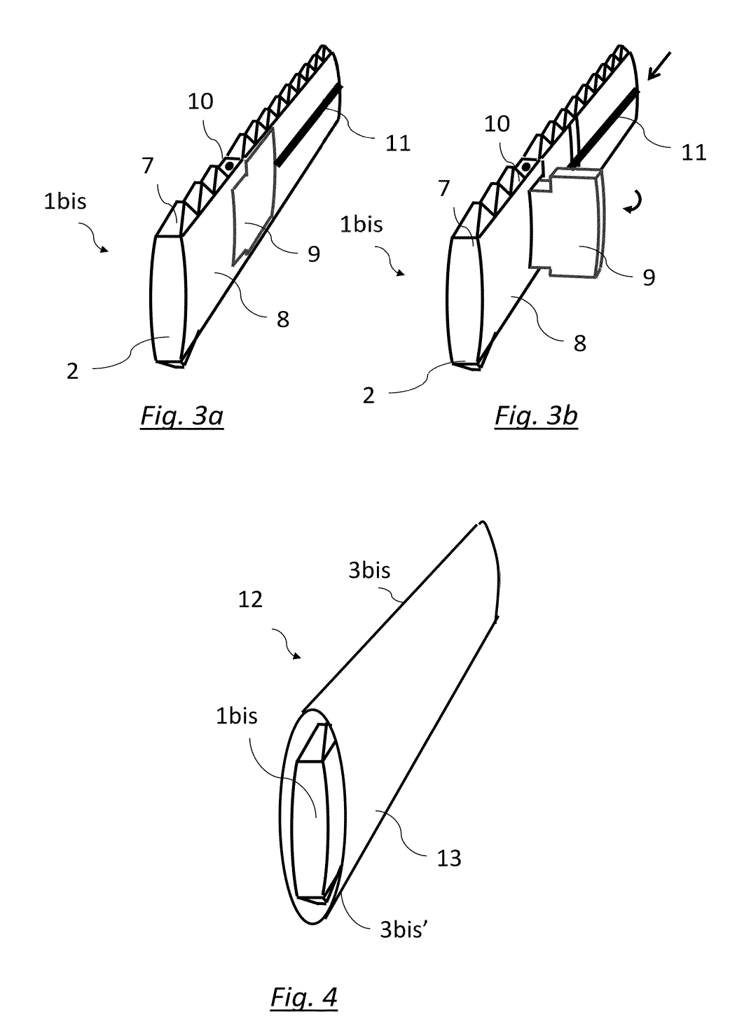

The invention may also have other variations: for instance, longitudinal body may only have one deployable flank on one of its lateral sides. The flanks may have cavities through them to enhance bone growth through the flanks towards the endplates. The longitudinal body may be relatively flexible in its longitudinal axis, so as to offer a laterally curved shape. As described in FIG. 3, edges 3, 3′ may have dents 7 to anchor into the endplates 6, 6′.

Another embodiment is represented in FIGS. 3a and 3b, where a spacer 1bis has a longitudinal body 2, with one flank 8 with a crutch 9 that is linked to the longitudinal body 2 by a hinge 10. This crutch 9 may be deployed laterally by rotation following an arc around hinge 10, which prevents the spacer 1 bis from toppling laterally once it is in its final position. FIG. 3b represents spacer 1bis with its deployed crutch 9. Deployment may be completed through any technical means, such as by a push rod 11 which slides along the posterior portion of flank 8 of longitudinal body 2.

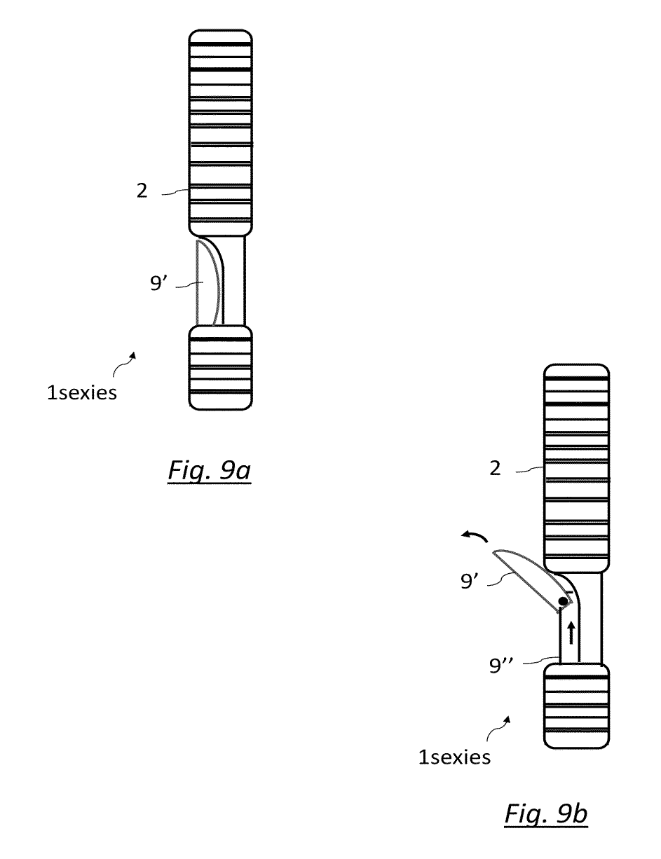

FIGS. 9a and 9b represent a variation, where spacer 1sexies has a crutch made of two portions 9′, 9″ liked together by a hinge. Crutch part 9′ may be deployed by the pushing of crutch part 9″ sliding along the side of longitudinal body 2. That surface has a widening cross-section in gradients: as crutch part 9′ slides against these changing gradients, its trajectory changes and crutch 9′ is pushed laterally off the longitudinal axis of longitudinal body. Crutch part 9′ may also have a side surface in gradients to enhance the change in trajectory when sliding out. There are other technical means to deploy a slideable crutch within the scope of this invention.

Another embodiment of the invention is represented in FIG. 4. It is a system 12 combining a spacer 1 or 1 bis and a sheath 13, the cross section of which is oval, ellipsoidal or in the shape of a race-track. The sheath has edges 3bis, 3bis′ at least on the length of its anterior portion. Spacer 1bis is fixed to an inserter (not represented) at its posterior part.

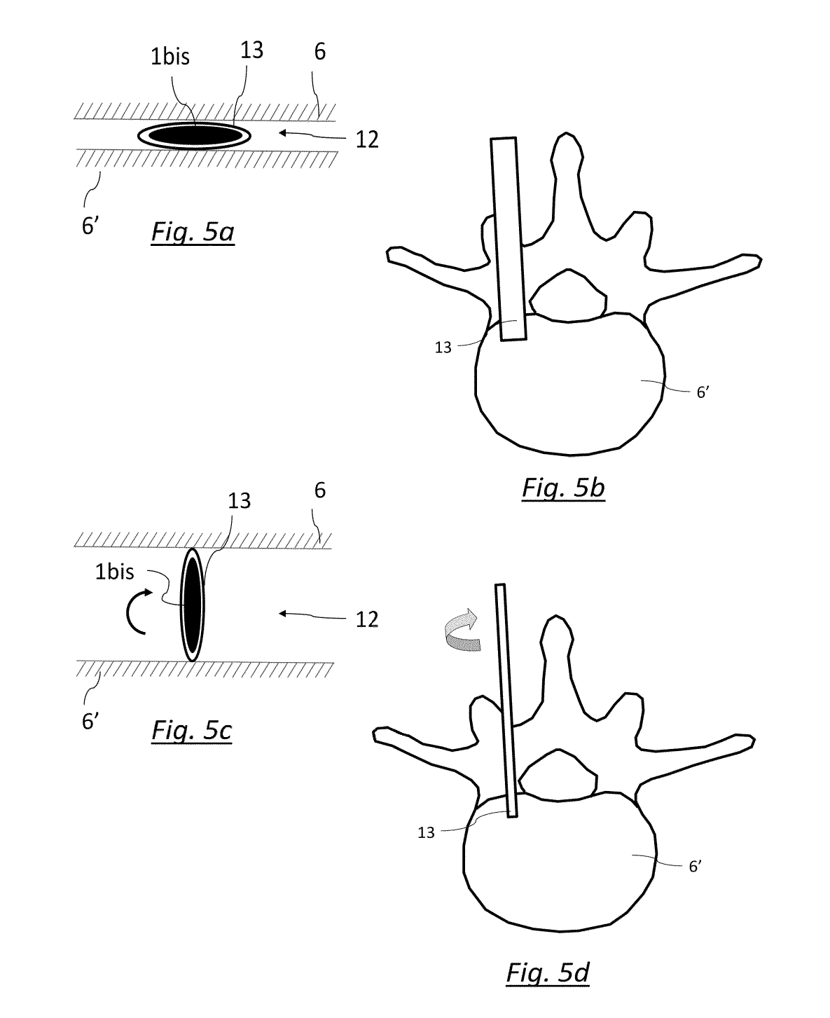

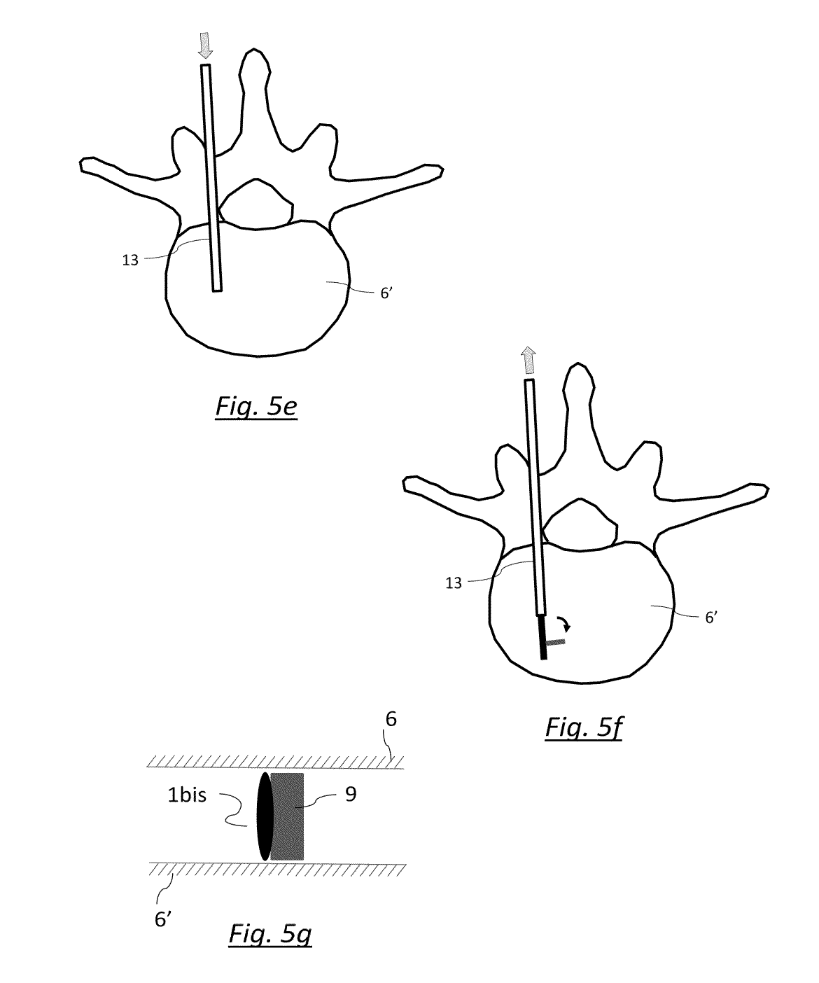

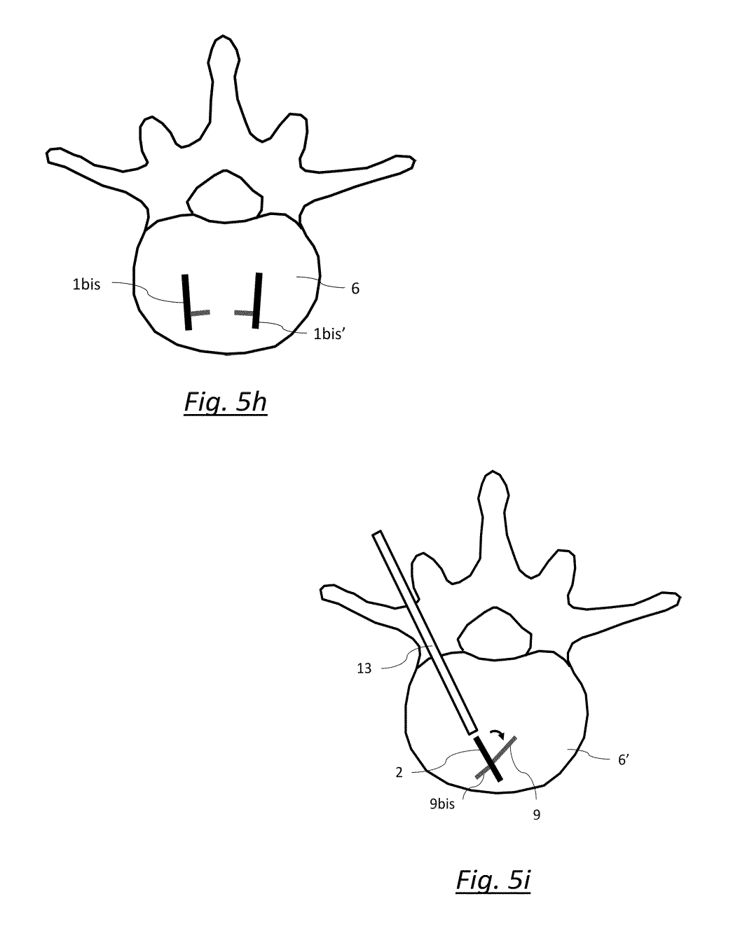

The method of insertion of system 12 is similar to the one described for the other embodiment represented in FIGS. 2a to 2d. The method for system 12 is described in FIGS. 5a to 5g. FIG. 5a represents the insertion of the tip of the sheath 13 by its smallest dimension between slightly distracted adjacent vertebrae 6, 6′. Spacer 1 or 1b, is housed inside the sheath 13, and has no contact with the vertebrae 6, 6′. FIG. 5b is a view in axial plane of the surface of vertebra 6′ and of sheath 13 with its tip on the cortical rim of the vertebra. FIG. 5c represents a section view of the vertebral segment 6, 6′ and the tip of the sheath 13 with its higher cross-section parallel to the axis of the spine, after a lateral rotation of the sheath 13 of 90°: the space between vertebrae 6, 6′ has been distracted. FIG. 5d represents vertebra 6′ and sheath 13 during the same phase as in FIG. 5c: the sheath is in an upright position compared to the vertebra 6′. FIG. 5e depicts how sheath 13 is then pushed forward in this upright position in a straight trajectory between the vertebrae until the final location planned for spacer 1 bis. FIG. 5f shows how sheath 13 is removed backwards, and spacer 1 bis is maintained in position by holding inserter (not represented) until the sheath is totally removed from the intervertebral space. Crutch 9 is then deployed laterally by the push of rod 11 (not represented). FIG. 5g represents spacer 1bis without sheath with a deployed crutch 9. FIG. 5h, represents two spacers 1bis, 1bis′ with deployed crutches in the intervertebral space. Within the same system 12, a variation of the method consists of not housing spacer 1bis within sheath 13, introduce the empty sheath 13 and perform all of the steps depicted in FIGS. 5a to 5e and only slide spacer 1 bis trough the inside of sheath 13 at the time of the step depicted in FIG. 5f. An additional method (not represented) consists of inserting sheath 13 in its smallest dimension until the final planned location of spacer 1bis and only rotate sheath 13 at such step; the step described in FIG. 5f is then the same.

Under another aspect of the invention (not represented in a figure), the sheath may also have a cross-section with a receding height so as to give a lordotic angle to that sheath, which may be appropriate to house a longitudinal body that has also a lordotic angle.

A variation of the invention is to insert a spacer with more than one crutches. FIG. 5i represents such a spacer for unilateral approaches (in the depicted case, transforaminal), where two lateral crutches 9, 9bis are deployed to increase the load surface of the spacer. Another variation (not represented) is to create a hinge between the anterior and posterior parts of the longitudinal body, in order to promote two different directional axes to that longitudinal body, and still deploy one or several crutches.

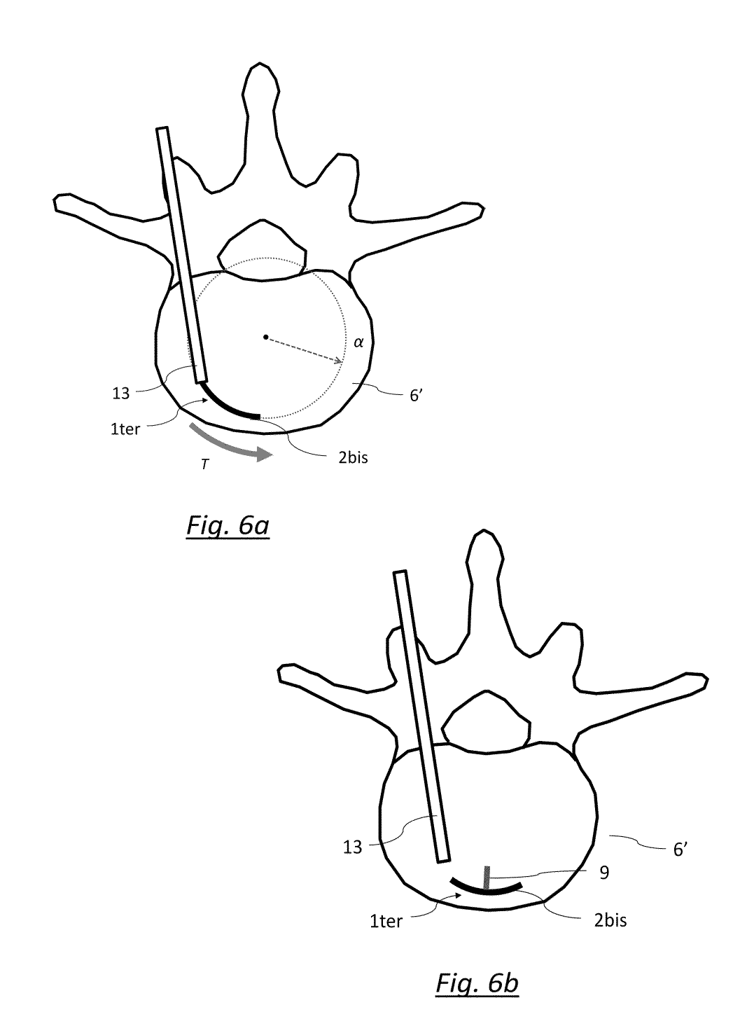

According to yet another variation of the invention, longitudinal body 2bis may be made of memory shape alloy as represented in FIGS. 6a and 6b. Sheath 13 is inserted as represented ins FIGS. 5a to 5e. FIG. 6a shows how spacer 1ter in memory shape alloy, is pushed out of sheath 13 through its tip, by pressure applied by the inserter (not represented) on the posterior part of spacer 1ter. Longitudinal body 2bis is freed from the lateral constraints of sheath 13, and takes its programmed curved shape, which corresponds to a segment of the rotational arc <<α>>, thus allowing spacer 1ter to follow trajectory <<T>>. FIG. 6b represents spacer1ter in its final location and position (after deployment of crutch 9). A variation of this invention would be to apply inflatable flanks, as described in the first embodiment) to such memory shape spacer.

FIGS. 5a to 5i and 6a and 6b represent spacers inserted by postero-lateral and unilateral transforaminal approaches. The method and system described herein applies to all relevant surgical approaches, such as the lateral or antero-lateral approaches.

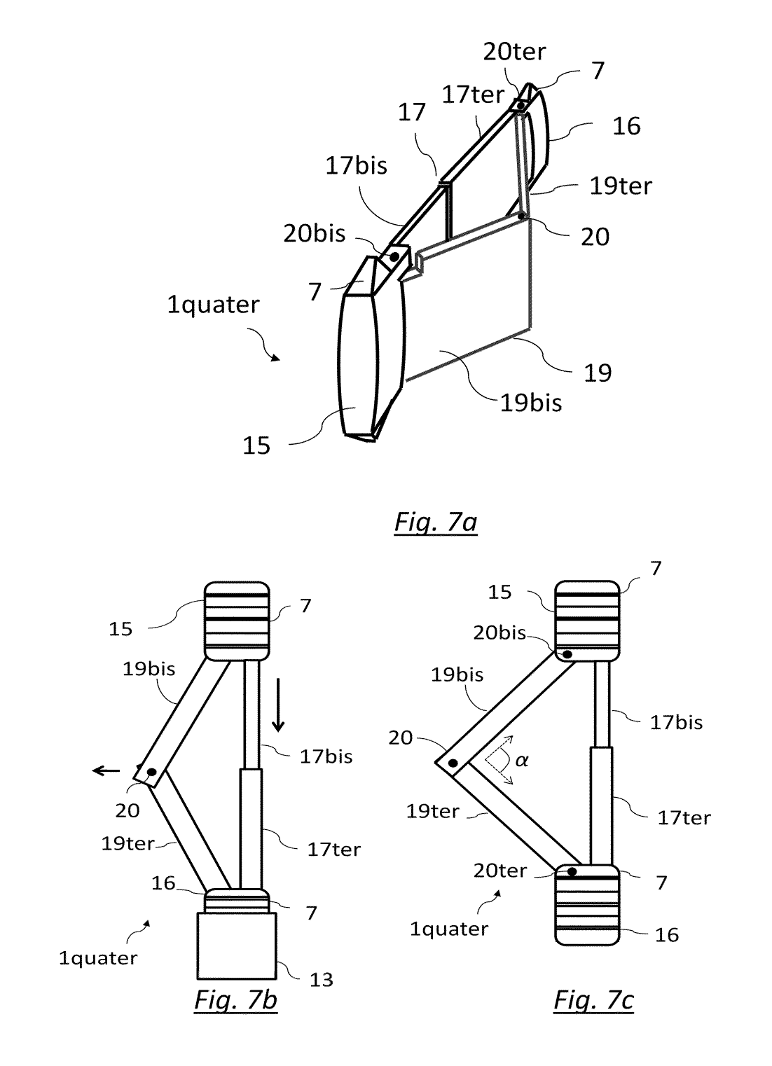

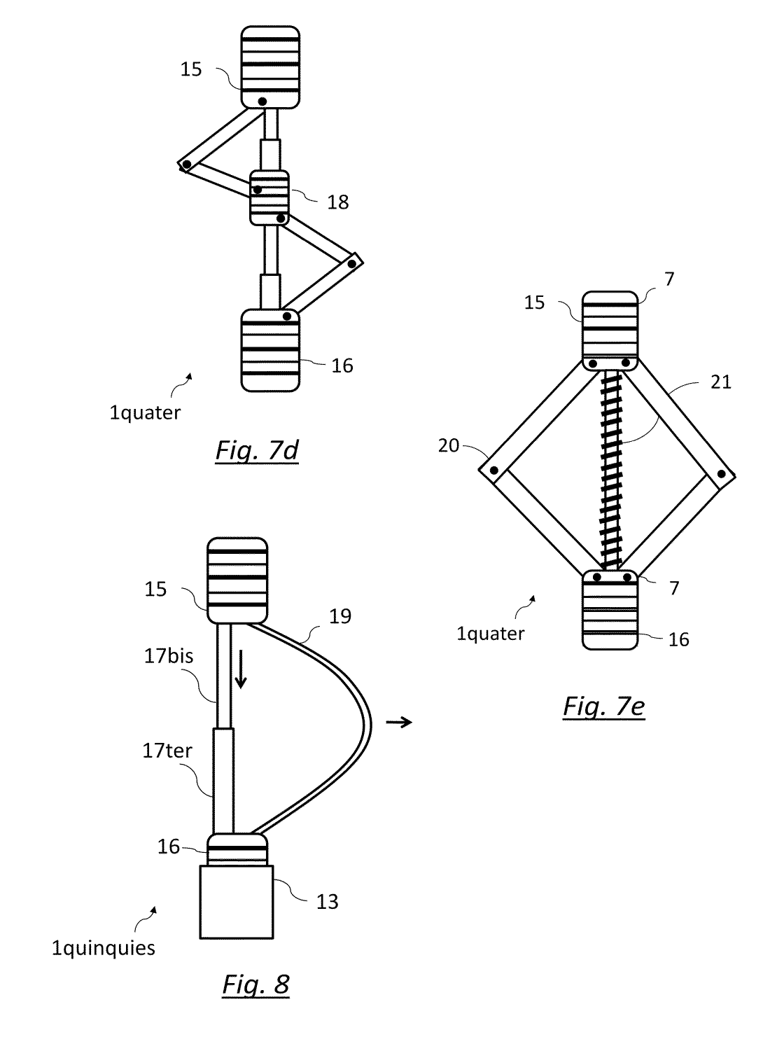

Another embodiment is described in FIGS. 7a, 7b and 7c. Spacer 1quater has an anterior portion 15 with dents 7 and a posterior portion 16 also with dents 7. Spacer 1quater has one first flank 17 with two telescopic sliding parts 17bis, 17ter, each solidly connected to anterior portion 15 and posterior portion 16, respectively. Telescopic parts 17bis, 17ter may slide one aside or inside of the other. Spacer 1quater has a second flank 19 comprising of at least two parts 19bis, 19ter with a hinge 20 between them, and each connected by another hinge 20bis, 20ter to anterior portion 15 and posterior portion 16, respectively. Spacer 1quater is inserted into the intervertebral space by a system including a sheath 13 according to the method described in FIGS. 5a to 5e. When sheath 13 is removed during the step pictured in FIG. 5d, spacer 1quater is compressed in its longitudinal axis, which brings anterior portion 15 and posterior portion 16 closer to one another. This compression causes telescopic parts 17bis, 17ter of flank 17 to slide one against the other (or one inside of the other), and the two parts 19bis, 19ter of the second flank 19 to pivot one relative to the other around hinge 20. This is possible because of hinges 20bis and 20ter connecting anterior portion 15 and posterior portion 16, respectively; parts 19bis and 19ter open an angle <<α>> around hinge 20. The compression of anterior portion 15 relative to posterior portion 16 of the spacer 1quater, and the maintenance of parts 19bis, 19ter in a deployed position relative to the longitudinal axis of spacer 1quater and to first flank 17, and crystallized in angle <<α>>, may be achieved by the turning of a conveying screw 21 connected between anterior portion 15 and posterior portion 16 or pulling and locking a cable connecting said portions 15, 16. Conveyor screw 21 or cable may be positioned within or outside telescopic parts 17bis, 17ter. A variation (not represented) is to replace hinge 20 by a part embedded in flank 19 made of flexible material or a spring.

Another embodiment (not represented) is to replace hinges 20, 20bis and/or 20ter by articulations with degrees of mobility in several different axes, in order to offer permanent mobility to the vertebral segment instead of seeking fusion, thus serving as prosthetic implant.

FIG. 7d represents a variation, with spacer 1quater having an anterior portion 15, a posterior portion 16 and a median portion 18, such three parts being connected by two pairs of telescopic flanks and of deployable flanks according to the same embodiment as described in FIGS. 7a to 7c. FIG. 7e represents another variation of spacer 1quater, whose two symmetric flanks deploy laterally as a result of the narrowing of the distance between anterior portion 15 and posterior portion 16, which is achieved by the screwing of the conveyor screw 21.

FIG. 8 represents another embodiment where spacer 1quinquies has one flank 19quater which has a cross-section like a thick ribbon, with a long axis substantially longer than its short axis, which provides flexibility in the direction perpendicular to the long axis. Flank 19quater is typically made of flexible material, but may also be or rigid material, such as metal. Flank 19quater deploys when anterior portion 15 and posterior portion 16 are brought closer together with the same result as in FIGS. 7a to 7c. A variation of this embodiment (not represented) is to replace flank 19quater with a longitudinal element made of flexible material, the cross section of which may be round, square, rectangular, oval, ellipsoidal or in the shape of a race-track.

A variation compared to the embodiment represented in FIGS. 9a and 9b (not represented) consists in deploying sliding crutch 9′ with the narrowing of the space between anterior portion 15 and posterior portion 16 as described in the embodiment in FIGS. 7a to 7c.

It goes without saying that certain characteristics of one embodiment may be substituted or added to characteristics of another embodiment.

1. Intervertebral spacer 1, 1bis, 1ter, 1quater, 1quinquies, 1sexies being a longitudinal body 2 with a cross-section that has a long axis and a short axis, at least one component of which may be deployed following an axis that is essentially perpendicular to the longitudinal body's long axis and to the axis of the spine.

2. The intervertebral spacer as in claim 1, wherein the deployment occurs by inflation of at least one flank 4, 4′.

3. The intervertebral spacer as in claim 1, wherein the deployment occurs with at least one crutch 9, 9′.

4. The intervertebral spacer as in claim 3, wherein the crutch 9 is deployed by rotation around the axis of a hinge 10.

5. The intervertebral spacer as in claim 3, wherein the crutch 9′ is deployed by sliding against the longitudinal body 2.

6. The intervertebral spacer as in claim 1, wherein the deployment of the component results from the compression between the anterior portion 15 and the posterior portion 16 of the spacer 1quater.

7. The intervertebral spacer as in claim 6, wherein the deploying component is one flank 19 made of two parts 19bis, 19ter connected by a hinge or an articulation.

8. The intervertebral spacer as in claim 6, wherein the deploying component is one flexible flank 19quater.

9. System 12 of a hollow instrument 13 with a cross-section that has a long axis and a short axis containing an intervertebral implant, wherein the instrument has continuous edges 3bis and 3bis′ at least on its distal portion.
